(19) United States
(12) Patent Application Publication
(10) Pub. No.: US 2023/0000470 A1
(43) Pub. Date: Jan. 5, 2023

(54) ULTRASOUND DIAGNOSIS APPARATUS AND RECORDING MEDIUM

(71) Applicant: CANON MEDICAL SYSTEMS CORPORATION, Tochigi (JP)

(72) Inventor: Hiroki TAKAHASHI, Nasushiobara (JP)

(73) Assignee: CANON MEDICAL SYSTEMS CORPORATION, Tochigi (JP)

(21) Appl. No.: 17/809,391

(22) Filed: Jun. 28, 2022

(30) Foreign Application Priority Data
Jun. 30, 2021 (JP) 2021-108672

(51) Int. Cl. A61B 8/08; A61B 8/00; G01S 15/89; G01S 7/52

(52) U.S. Cl. CPC A61B 8/5269; A61B 8/4488; A61B 8/5207; G01S 15/8911; G01S 7/52095

(57) ABSTRACT

An ultrasound diagnosis apparatus according to an embodiment is configured to implement an ultrasound beamforming method by which, among a plurality of reception signals output from a plurality of elements, reception signals from mutually-different elements are multiplied by each other, so that signals obtained as results of the multiplications are added together. The ultrasound diagnosis apparatus according to the embodiment includes processing circuitry. The processing circuitry is configured to calculate a weight coefficient on the basis of a correlation between the multiplied reception signals. The processing circuitry is configured to apply the weight coefficient to the signals obtained as the results of the multiplications.

### CROSS-REFERENCE TO RELATED APPLICATIONS

This application is based upon and claims the benefit of priority from Japanese Patent Application No. 2021-108672, filed on Jun. 30, 2021; the entire contents of which are incorporated herein by reference.

### FIELD

Embodiments described herein relate generally to an ultrasound diagnosis apparatus and a recording medium.

### BACKGROUND

As a reception beamforming technique implemented by ultrasound diagnosis apparatuses, a method called “Delay and Sum (DAS)” is commonly used. In recent years, various methods different from the DAS method have been proposed. Examples of known methods include “minimum variance beamforming”, “coherence factor imaging”, and “Delay Multiply and Sum Beamforming (DMAS)”. Among these methods, according to a DMAS method, multiplication and addition are performed on signals output from mutually-different elements (piezoelectric transducer elements or piezoelectric elements).

As explained above, according to the DMAS method, the signals output from the mutually-different elements are multiplied by each other. Accordingly, in ultrasound image data obtained by implementing the DMAS method, output signals exhibit a more sensitive reaction to a time lag (a time difference or a phase difference) between signals than in ultrasound image data obtained by using the DAS method. The more sensitive reaction appears as a larger amplitude fluctuation of speckle patterns (speckle noise), for example. In other words, in the ultrasound image data obtained by implementing the DMAS method, the speckle patterns appear in a more emphasized manner than in the ultrasound image data obtained by implementing the DAS method. As explained herein, because the signals are multiplied by each other according to the DMAS method, contrast resolution is lowered due to a larger amplitude fluctuation of speckle echoes.

### BRIEF DESCRIPTION OF THE DRAWINGS

FIG. 1 is a block diagram illustrating an exemplary configuration of an ultrasound diagnosis apparatus according to a first embodiment;

FIG. 2 is a diagram illustrating an exemplary configuration of a beam former included in a reception circuit according to the first embodiment and configured to implement beamforming using a DMAS method;

FIG. 3 is a flowchart illustrating an example of a flow in a process performed by the beam former implementing the DMAS method according to the first embodiment;

FIG. 4 is a drawing illustrating an example of an ultrasound image based on ultrasound image data obtained by using a conventional DMAS method;

FIG. 5 is a drawing illustrating an example of an ultrasound image based on ultrasound image data generated by the ultrasound diagnosis apparatus according to the first embodiment;

FIG. 6 is a diagram illustrating an exemplary configuration of a beam former included in a reception circuit according to a modification example of the first embodiment and configured to implement beamforming using the DMAS method;

FIG. 7 is a diagram illustrating an example of a partial configuration of a beam former according to a second embodiment;

FIG. 8 is a drawing illustrating an example of an ultrasound image based on ultrasound image data generated by an ultrasound diagnosis apparatus according to the second embodiment;

FIG. 9 is a diagram illustrating an example of a partial configuration of a beam former according to a third embodiment; and

FIG. 10 is a diagram illustrating an example of a partial configuration of a beam former according to a fourth embodiment.

### DETAILED DESCRIPTION

One of the problems to be solved by the embodiments disclosed in the present specification and drawings is to inhibit the occurrence of speckle patterns when a method involving multiplication and addition of signals is used as a beamforming method. However, the problems to be solved by the embodiments disclosed in the present specification and drawings are not limited to the abovementioned problem. It is also possible to consider problems corresponding to advantageous effects achieved by the configurations in the embodiments described below as other problems.

An ultrasound diagnosis apparatus according to an embodiment is configured to implement an ultrasound beamforming method by which, among a plurality of reception signals output from a plurality of elements, reception signals from mutually-different elements are multiplied by each other, so that signals obtained as results of the multiplications are added together. The ultrasound diagnosis apparatus according to the embodiment includes processing circuitry. The processing circuitry is configured to calculate a weight coefficient on the basis of a correlation between the multiplied reception signals. The processing circuitry is configured to apply the weight coefficient to the signals obtained as the results of the multiplications.

Exemplary embodiments and modification examples of an ultrasound diagnosis apparatus and a program will be explained below, with reference to the accompanying drawings.

### First Embodiment

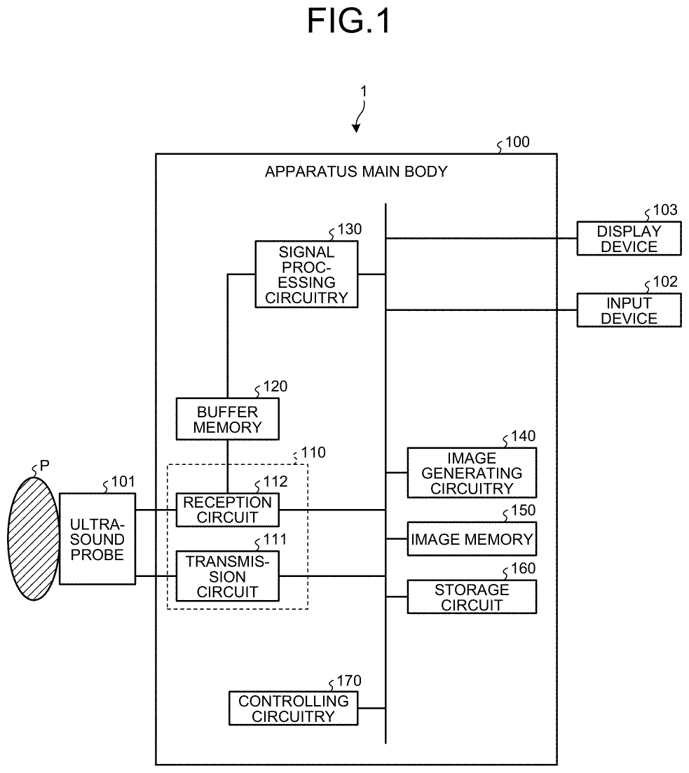

FIG. 1 is a block diagram illustrating an exemplary configuration of an ultrasound diagnosis apparatus 1 according to a first embodiment. As illustrated in FIG. 1, the ultrasound diagnosis apparatus 1 according to the first embodiment includes an apparatus main body 100, an ultrasound probe 101, an input device 102, and a display device 103. In the first embodiment, the ultrasound diagnosis apparatus 1 is configured to implement a DMAS method, which is an ultrasound beamforming method by which, among a plurality of reception signals output from a plurality of elements in the ultrasound probe 101, reception signals from mutually-different elements are multiplied by each other so that signals obtained as the results of the multiplications are added together.

For example, the ultrasound probe 101 includes a plurality of elements (piezoelectric transducer elements or piezoelectric elements). The plurality of elements are configured to generate an ultrasound wave on the basis of a drive signal supplied from a transmission circuit 111 of a transmission and reception circuit 110 included in the apparatus main body 100. More specifically, as a result of the transmission circuit 111 applying voltage (transmission drive voltage) thereto, the plurality of elements are configured to generate the ultrasound wave having a waveform corresponding to the transmission drive voltage. The waveform of the transmission drive voltage indicated by the drive signal is the waveform of the voltage applied to the plurality of elements. In other words, the ultrasound probe 101 is configured to transmit the ultrasound wave corresponding to the magnitude of the applied transmission drive voltage. Further, the ultrasound probe 101 is configured to receive a reflected wave arriving from an examined subject (hereinafter, “patient”) P, to convert the reflected wave into a reception signal (a reflected-wave signal) being an electrical signal, and to output the reception signal to the apparatus main body 100. Further, the ultrasound probe 101 includes, for example, a matching layer provided for the elements, a backing member configured to prevent ultrasound waves from propagating rearward from the elements, and the like. In this situation, the ultrasound probe 101 is detachably connected to the apparatus main body 100.

When the ultrasound wave is transmitted from the ultrasound probe 101 to the patient P, the transmitted ultrasound wave is repeatedly reflected on a surface of discontinuity of acoustic impedances at a tissue in the body of the patient P, so as to be received as the reflected wave by the plurality of elements included in the ultrasound probe 101. The amplitude of the received reflected wave is dependent on the difference between the acoustic impedances on the surface of discontinuity on which the ultrasound wave is reflected. Further, when a transmitted ultrasound pulse is reflected on the surface of a moving blood flow, a cardiac wall, or the like, the reflected wave is, due to the Doppler effect, subject to a frequency shift, depending on a velocity component of the moving members with respect to the ultrasound wave transmission direction. Further, the ultrasound probe 101 is configured to output the reception signal to a reception circuit 112 (explained later) of the transmission and reception circuit 110.

The ultrasound probe 101 is provided so as to be attachable to and detachable from the apparatus main body 100. When a two-dimensional region in the patient P is to be scanned (a two-dimensional scan), for example, an operator connects, as the ultrasound probe 101, a one-dimensional (1D) array probe in which the plurality of elements are arranged in a single row, to the apparatus main body 100. Examples of different types of the 1D array probe include linear ultrasound probes, convex ultrasound probes, and sector ultrasound probes. Further, when a three-dimensional region in the patient P is to be scanned (a three-dimensional scan), for example, the operator connects, as the ultrasound probe 101, a mechanical four-dimensional (4D) probe or a two-dimensional (2D) array probe, to the apparatus main body 100. The mechanical 4D probe is capable of performing a two-dimensional scan by using the plurality of elements arranged in a single row like in the 1D array probe and is also capable of performing a three-dimensional scan by swinging the plurality of elements at a predetermined angle (a swing angle). Further, the 2D array probe is capable of performing a three-dimensional scan by using the plurality of elements arranged in a matrix formation and is also capable of performing a two-dimensional scan by transmitting an ultrasound wave in a converged manner.

The input device 102 is realized, for example, by using input means such as a mouse, a keyboard, a button, a panel switch, a touch command screen, a foot switch, a trackball, a joystick, and/or the like. The input device 102 is configured to receive various types of setting requests from the operator of the ultrasound diagnosis apparatus 1 and to transfer the received various types of setting requests to the apparatus main body 100.

The display device 103 is configured, for example, to display a Graphical User Interface (GUI) used by the operator of the ultrasound diagnosis apparatus 1 for inputting the various types of setting requests via the input device 102 and to display an ultrasound image based on ultrasound image data generated in the apparatus main body 100, and the like. The display device 103 is realized by using a liquid crystal monitor, a Cathode Ray Tube (CRT) monitor, or the like.

The apparatus main body 100 is configured to generate the ultrasound image data on the basis of the reception signal transmitted thereto from the ultrasound probe 101. In this situation, the ultrasound image data is an example of image data. The apparatus main body 100 is capable of generating two-dimensional ultrasound image data on the basis of the reception signal corresponding to a two-dimensional region of the patient P and having been transmitted thereto from the ultrasound probe 101. Further, the apparatus main body 100 is capable of generating three-dimensional ultrasound image data on the basis of the reception signal corresponding to a three-dimensional region of the patient P and having been transmitted thereto from the ultrasound probe 101. As illustrated in FIG. 1, the apparatus main body 100 includes the transmission and reception circuit 110, a buffer memory 120, signal processing circuitry 130, image generating circuitry 140, an image memory 150, a storage circuit 160, and controlling circuitry 170.

The transmission and reception circuit 110 is configured, under control of the controlling circuitry 170, to cause the ultrasound probe 101 to transmit the ultrasound wave and to cause the ultrasound probe 101 to receive the reflected wave of the ultrasound wave. In other words, the transmission and reception circuit 110 is configured to perform the scan via the ultrasound probe 101. The scan may be referred to as scanning, ultrasound scanning, or an ultrasound scan. The transmission and reception circuit 110 is an example of a transmission and reception unit. The transmission and reception circuit 110 includes the transmission circuit 111 and the reception circuit 112. The transmission circuit 111 is an example of a transmission unit, whereas the reception circuit 112 is an example of a reception unit.

Under the control of the controlling circuitry 170, the transmission circuit 111 is configured to cause the ultrasound wave to be transmitted from the ultrasound probe 101. The transmission circuit 111 includes a rate pulser generating circuit, a transmission delay circuit, and a transmission pulser. The transmission circuit 111 is configured to supply the drive signal to the ultrasound probe 101. When a two-dimensional region in the patient P is to be scanned, the transmission circuit 111 is configured to cause the ultrasound probe 101 to transmit an ultrasound beam for scanning the two-dimensional region. In contrast, when a three-dimensional region in the patient P is to be scanned, the transmission circuit 111 is configured to cause the ultrasound probe 101 to transmit an ultrasound beam for scanning the three-dimensional region.

Under control of the controlling circuitry 170, the rate pulser generating circuit is configured to repeatedly generate a rate pulse for forming a transmission ultrasound wave (a transmission beam) at a predetermined rate frequency (a Pulse Repetition Frequency [PRF]). As a result of the rate pulse being routed through the transmission delay circuit, voltage is applied to the transmission pulser while having mutually-different transmission delay time periods. For example, the transmission delay circuit is configured to apply the transmission delay time period that corresponds to each of the elements and is required to converge the ultrasound wave generated from the ultrasound probe 101 into the form of a beam and to determine transmission directionality, to the rate pulses generated by the rate pulser generating circuit. The transmission pulser is configured to supply the drive signal (a drive pulse) to the ultrasound probe 101 with timing based on the rate pulses. In other words, the transmission pulser is configured to apply the voltage (the transmission drive voltage) having the waveform indicated by the drive signal to the ultrasound probe 101, with the timing based on the rate pulses. In this situation, the transmission delay circuit is configured to arbitrarily adjust the transmission direction of the ultrasound wave arriving from the surfaces of the elements, by varying the transmission delay time periods applied to the rate pulses.

The drive pulse travels from the transmission pulser and reaches the elements provided in the ultrasound probe 101 via a cable and is subsequently converted at the elements from an electrical signal into mechanical vibration. In other words, as a result of the voltage being applied to the elements, the elements are configured to mechanically vibrate. The ultrasound wave generated by the mechanical vibration is transmitted to the inside of the patient's body (the inside of the patient P). In this situation, the ultrasound waves having the mutually-different transmission delay time periods in correspondence with the elements are converged so as to propagate in a predetermined direction.

Further, under the control of the controlling circuitry 170, the transmission circuit 111 has a function capable of instantaneously changing the transmission frequency, the transmission drive voltage, and the like for executing a predetermined scan sequence. In particular, the capability to change the transmission drive voltage is realized by a transmission circuit of a linear amplifier type capable of instantaneously switching the value of the transmission drive voltage or a mechanism configured to electrically switch between a plurality of power source units.

The reflected wave of the ultrasound wave transmitted by the ultrasound probe 101 reaches the elements provided inside the ultrasound probe 101 and is subsequently converted at the elements from mechanical vibration into an electrical signal (the reception signal), so that the reception signal is input to the reception circuit 112. The reception circuit 112 includes a preamplifier, an Analog-to-Digital (A/D) converter, a quadrature detection circuit, a beam former implementing the DMAS method (explained later), and the like and is configured to generate reflected-wave data (reception data) by performing various types of processes on the reception signal transmitted thereto from the ultrasound probe 101. Further, the reception circuit 112 is configured to store the generated reflected-wave data into the buffer memory 120.

The pre-amplifier is configured to amplify the reception signal with respect to each of the channels and to perform a gain adjustment (a gain correction) thereon. Further, the A/D converter is configured to convert the gain-corrected reception signal into a digital signal by performing an A/D conversion on the gain-corrected reception signal. The quadrature detection circuit is configured to convert the reception signal converted to the digital signal into an In-phase signal (an I signal) and a Quadrature-phase signal (a Q signal) in a base band. After that, the quadrature detection circuit is configured to transmit the I signal and the Q signal (IQ signals) to the beam former implementing the DMAS method. Further, the beam former is configured to perform the beamforming implementing the DMAS method on the IQ signals, so that data resulting from the beamforming implementing the DMAS method is stored, as the reflected-wave data, into the buffer memory 120. The beam former implementing the DMAS method will be explained later.

The reception circuit 112 is configured to generate two-dimensional reflected-wave data from two-dimensional reception signal transmitted thereto from the ultrasound probe 101. Further, the reception circuit 112 is configured to generate three-dimensional reflected-wave data from three-dimensional reception signal transmitted thereto from the ultrasound probe 101.

The buffer memory 120 is a memory configured to temporarily store therein the reflected-wave data generated by the transmission and reception circuit 110. For example, the buffer memory 120 is configured so as to be able to store therein the reflected-wave data corresponding to a predetermined number of frames. Further, when reflected-wave data corresponding to one frame is newly generated by the reception circuit 112 while the buffer memory 120 has stored therein the reflected-wave data corresponding to the predetermined number of frames, the buffer memory 120 discards reflected-wave data corresponding to one of the frames generated earliest and stores therein the newly-generated reflected-wave data corresponding to the one frame, under the control of the reception circuit 112. For example, the buffer memory 120 is realized by using a semiconductor memory element such as a Random Access Memory (RAM) or a flash memory.

The signal processing circuitry 130 is configured to read the reflected-wave data from the buffer memory 120, to perform various types of signal processing processes on the read reflected-wave data, and to output the reflected-wave data on which the various types of signal processing processes have been performed to the image generating circuitry 140 as B-mode data or Doppler data. For example, the signal processing circuitry 130 is realized by using one or more processors. The signal processing circuitry 130 is an example of a signal processing unit.

For example, every time reflected-wave data corresponding to one frame is newly stored in the buffer memory 120, the signal processing circuitry 130 is configured to read the reflected-wave data corresponding to the one frame newly stored in the buffer memory 120. After that, by performing the various types of signal processing processes on the read reflected-wave data corresponding to the one frame, the signal processing circuitry 130 is configured to newly generate B-mode data or Doppler data corresponding to the one frame. After that, every time B-mode data or Doppler data corresponding one frame is generated, the signal processing circuitry 130 is configured to output the newly-generated B-mode data or Doppler data corresponding to the one frame to the image generating circuitry 140. In the following sections, an example of the various types of signal processing processes performed by the signal processing circuitry 130 will be explained.

For example, by performing a quadrature detection, a logarithmic amplification, an envelope detection process, and the like on the reflected-wave data read from the buffer memory 120, the signal processing circuitry 130 is configured to generate B-mode data in which the signal intensity (amplitude intensity) at each sampling point is expressed as a degree of brightness. For example, the signal processing circuitry 130 is configured to output the generated B-mode data to the image generating circuitry 140.

Further, by performing a frequency analysis on the reflected-wave data read from the buffer memory 120, the signal processing circuitry 130 is configured to extract movement information of moving members (blood flows, tissues, contrast agent echo components, etc.) based on the Doppler effect from the reflected-wave data and to generate Doppler data indicating the extracted movement information. For example, the signal processing circuitry 130 is configured to generate the Doppler data indicating the extracted movement information of the moving members, by extracting, as the movement information of the moving members, an average velocity value, an average dispersion value, an average power value, and the like with respect to multiple points. The signal processing circuitry 130 is configured to output the generated Doppler data to the image generating circuitry 140.

By using the functions of the signal processing circuitry 130 described above, the ultrasound diagnosis apparatus 1 is capable of implementing a color Doppler method which may be called a Color Flow Mapping (CEM) method. According to the color flow mapping method, ultrasound wave transmission and reception is performed multiple times on a plurality of scanning lines. Further, according to the color flow mapping method, a signal (a blood flow signal) derived from a blood flow is extracted from data sequences corresponding to mutually the same position, while suppressing signals (clutter signals) derived from stationary tissues or slow-moving tissues, by applying a Moving Target Indicator (MTI) filter to the data sequences corresponding to mutually the same position. Further, according to the color flow mapping method, blood flow information, such as velocity of the blood flow, dispersion of the blood flow, power of the blood flow, and the like, is estimated on the basis of the blood flow signal. The signal processing circuitry 130 is configured to output color image data indicating the blood flow information estimated by implementing the color flow mapping method, to the image generating circuitry 140. The color image data is an example of Doppler data.

The signal processing circuitry 130 is capable of processing both types of the reflected-wave data, namely the two-dimensional reflected-wave data and the three-dimensional reflected-wave data.

The image generating circuitry 140 is configured to generate the ultrasound image data from the B-mode data or the Doppler data output from the signal processing circuitry 130. The image generating circuitry 140 is realized by using one or more processors.

For example, the image generating circuitry 140 is configured to generate two-dimensional B-mode image data in which intensities of the reflected wave are expressed with brightness levels, from two-dimensional B-mode data generated by the signal processing circuitry 130. Further, the image generating circuitry 140 is configured to generate two-dimensional Doppler image data in which the movement information or the blood flow information is visualized in an image, from two-dimensional Doppler data generated by the signal processing circuitry 130. In this situation, the two-dimensional Doppler image data obtained by visualizing the movement information in the image is velocity image data, dispersion image data, power image data, or image data combining together any of these types of image data.

In this situation, generally speaking, the image generating circuitry 140 is configured to convert (by performing a scan convert process) a scanning line signal sequence from an ultrasound scan into a scanning line signal sequence in a video format used by television, for example, and to generate display-purpose ultrasound image data. For example, the image generating circuitry 140 is configured to generate the display-purpose ultrasound image data by performing a coordinate transformation process compliant with an ultrasound scanning mode used by the ultrasound probe 101 on the data output from the signal processing circuitry 130. Further, as various types of image processing processes besides the scan convert process, the image generating circuitry 140 is configured to perform, for example, an image processing process (a smoothing process) to re-generate an average brightness value image, an image processing process (an edge enhancement process) that uses a differential filter inside an image, or the like, by using a plurality of image frames resulting from the scan convert process. Also, the image generating circuitry 140 is configured to combine text information of various types of parameters, scale graduations, body marks, and the like with the ultrasound image data.

Further, the image generating circuitry 140 is configured to generate three-dimensional B-mode image data by performing a coordinate transformation process on three-dimensional B-mode data generated by the signal processing circuitry 130. Further, the image generating circuitry 140 is configured to generate three-dimensional Doppler image data by performing a coordinate transformation process on three-dimensional Doppler data generated by the signal processing circuitry 130. In other words, the image generating circuitry 140 is configured to generate the “three-dimensional B-mode image data and three-dimensional Doppler image data” as “three-dimensional ultrasound image data (volume data)”. Further, the image generating circuitry 140 is configured to perform various types of rendering processes on the volume data, so as to generate various types of two-dimensional image data used for displaying the volume data on the display device 103.

Examples of the rendering processes performed by the image generating circuitry 140 include a process of generating Multi Planar Reconstruction (MPR) image data from the volume data by using a Multi Planar Reconstruction (MPR) method. Further, other examples of the rendering processes performed by the image generating circuitry 140 include a Volume Rendering (VR) process by which two-dimensional image data reflecting three-dimensional information is generated. The image generating circuitry 140 is an example of an image generating unit.

The B-mode data and the Doppler data are each ultrasound image data before the scan convert process. The data generated by the image generating circuitry 140 is the display-purpose ultrasound image data after the scan convert process. The B-mode data and the Doppler data may be referred to as raw data.

The image memory 150 is a memory configured to store therein various types of image data generated by the image generating circuitry 140. Further, the image memory 150 is also configured to store therein the data generated by the signal processing circuitry 130. An operator is able to invoke the B-mode data and the Doppler data stored in the image memory 150 after a diagnosis process, for example. The invoked data serves as display-purpose ultrasound image data after being routed through the image generating circuitry 140. For example, the image memory 150 is realized by using a semiconductor memory element such as a Random Access Memory (RAN) or a flash memory, or a hard disk, an optical disk, or the like.

The storage circuit 160 is configured to store therein control programs for performing the scan (transmitting and receiving ultrasound waves), image processing processes, and display processes, as well as various types of data such as diagnosis information (e.g., patient IDs, medical doctors' observations, etc.), diagnosis protocols, and various types of body marks. Further, the storage circuit 160 may also be used for saving any of the data stored in the image memory 150, as necessary. For example, the storage circuit 160 is realized by using a semiconductor memory element such as a flash memory, or a hard disk, an optical disk, or the like.

The controlling circuitry 170 is configured to control the entirety of processes performed by the ultrasound diagnosis apparatus 1. More specifically, the controlling circuitry 170 is configured to control processes performed by the transmission circuit 111, the reception circuit 112, the signal processing circuitry 130, and the image generating circuitry 140, on the basis of the various types of setting requests input from the operator via the input device 102 and the various types of control programs and various types of data read from the storage circuit 160. Further, the controlling circuitry 170 is configured to control the display device 103 so as to display ultrasound images based on the display-purpose ultrasound image data stored in the image memory 150. For example, the controlling circuitry 170 is configured to control the display device 103 so as to display a B-mode image based on B-mode image data or a color image based on color image data. Further, the controlling circuitry 170 is configured to control the display device 103 so as to display a color image superimposed on a B-mode image. The display controlling circuitry 170 is an example of a display controlling unit or a controlling unit. For example, the controlling circuitry 170 is realized by using one or more processors. The ultrasound images are examples of images.

Further, the controlling circuitry 170 is configured to control the ultrasound scan, by controlling the ultrasound probe 101 via the transmission and reception circuit 110.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). One or more processors are configured to realize the functions by reading the programs saved in the storage circuit 160 and executing the read programs. Alternatively, instead of having the programs saved in the storage circuit 160, it is also acceptable to directly incorporate the programs into the circuits of the one or more processors. In that situation, the one or more processors are configured to realize the functions by reading and executing the programs incorporated in the circuits thereof. Further, the processors according to the present embodiments do not each necessarily have to be structured as a single circuit. It is also acceptable to structure one processor by combining together a plurality of independent circuits, so as to realize the functions thereof. Further, two or more of the pieces of circuitry (e.g., the signal processing circuitry 130, the image generating circuitry 140, and the controlling circuitry 170) illustrated in FIG. 1 may be integrated in a single processor so as to realize the functions thereof. In other words, the signal processing circuitry 130, the image generating circuitry 140, and the controlling circuitry 170 may be integrated into one piece of processing circuitry realized by the processor.

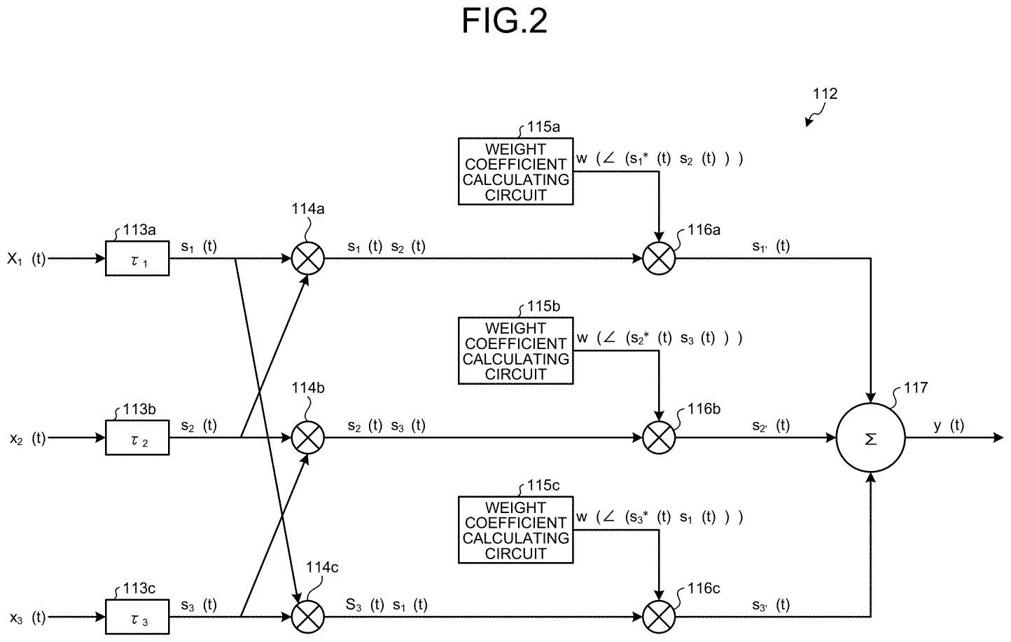

An overall configuration of the ultrasound diagnosis apparatus 1 according to the embodiment has thus been explained. Next, an exemplary configuration of the beam former included in the reception circuit 112 and configured to implement the beamforming using the DMAS method will be explained. FIG. 2 is a diagram illustrating the exemplary configuration of the beam former included in the reception circuit 112 according to the first embodiment and configured to implement the beamforming using the DMAS method. In the example in FIG. 2, for the sake of convenience in the explanation, the quantity of the elements in the ultrasound probe 101 is three, while one channel corresponds to each of the elements. However, the quantity of the elements in the ultrasound probe 101 does not have to be three and may be N (where N is a natural number). Further, one channel may correspond to two or more elements. Further, to identify the three elements, an element number n (where n=1, 2, 3) is assigned to each of the elements. For example, element number 1 is assigned to a first element; element number 2 is assigned to a second element; and element number 3 is assigned to a third element.

As illustrated in FIG. 2, the beam former in the reception circuit 112 includes three delay circuits 113a to 113c, three multipliers 114a to 114c, three weight coefficient calculating circuits 115a to 115c, three multipliers 116a to 116c, and an adder 117. The beam former may be realized by using a processor.

When not distinguished from one another, the three delay circuits 113a to 113c may be referred to as “delay circuits 113”. Similarly, when not distinguished from one another, the three multipliers 114a to 114c may be referred to as “multipliers 114”. When not distinguished from one another, the three weight coefficient calculating circuits 115a to 115c may be referred to as “weight coefficient calculating circuits 115”. When not distinguished from one another, the three multipliers 116a to 116c may be referred to as “multipliers 116”.

For example, with respect to each of the channels, a different one of the delay circuits 113, a different one of the multipliers 114, a different one of the weight coefficient calculating circuits 115, and a different one of the multipliers 116 are provided.

The expression xₙ(t) denotes IQ signals that are based on a reception signal output from element number n and that correspond to a time t. As illustrated in FIG. 2, IQ signals x₁(t) are input to the delay circuit 113a. The delay circuit 113a is configured to output the IQ signals x₁(t) delayed by a time period τ1. In other words, the delay circuit 113a is configured to output IQ signals s₁(t) obtained by delaying the IQ signals x₁(t) by the time period τ1, to the multiplier 114a, the multiplier 114c, the weight coefficient calculating circuit 115a, and the weight coefficient calculating circuit 115c. Alternatively, the xₙ(t) does not necessarily have to be IQ signals and may be a Radio Frequency (RF) signal having only a real number part.

Similarly, the delay circuit 113b is configured to delay IQ signals x₂(t) having been input thereto by a time period τ2 and to output IQ signals s₂(t) obtained by delaying the IQ signals x₂(t) by the time period τ2 to the multiplier 114a, the multiplier 114b, the weight coefficient calculating circuit 115a, and the weight coefficient calculating circuit 115b.

Further, the delay circuit 113c is configured to delay IQ signals x₃(t) having been input thereto by a time period 13 and to output IQ signals s₃(t) obtained by delaying the IQ signals x₃(t) by the time period τ3 to the multiplier 114b, the multiplier 114c, the weight coefficient calculating circuit 115b, and the weight coefficient calculating circuit 115c.

In the present example, the time period τ1 is a delay time period corresponding to a positional relationship between the position of the element identified with element number 1 and the position of a focal point. Further, the time period τ1 is also a delay time period simply corresponding to the position of the focal point. Similarly, the time period 12 is a delay time period corresponding to a positional relationship between the position of the element identified with element number 2 and the position of the focal point. The time period τ3 is a delay time period corresponding to a positional relationship between the position of the element identified with element number 3 and the position of the focal point. Further, the time period τ2 and the time period τ3 are each also a delay time period simply corresponding to the position of the focal point.

As explained above, the delay circuits 113 are configured to output the plurality of delayed signals that are delayed by applying the delay time periods corresponding to the positions of the focal point to the plurality of reception signals output from the plurality of elements in the ultrasound probe 101. The delay circuits 113 are each an example of a delay unit.

The multiplier 114a is configured to multiply the IQ signals s₁(t) by the IQ signals s₂(t). After that, the multiplier 114a is configured to output a signal s₁(t)s₂(t) obtained as the result of the multiplication, to the multiplier 116a.

Similarly, the multiplier 114b is configured to multiply the IQ signals s₂(t) by the IQ signals s₃(t) and to output a signal s₂(t)s₃(t) obtained as the result of the multiplication, to the multiplier 116b. Also, the multiplier 114c is configured to multiply the IQ signals s₃(t) by the IQ signals s₁(t) and to output a signal s₃(t)s₁(t) obtained as the result of the multiplication, to the multiplier 116c.

The weight coefficient calculating circuit 115a is configured to calculate a weight coefficient (a weight) to be applied to the signal s₁(t)s₂(t). In the following sections, a specific example of a method for calculating the weight coefficient to be applied to the signal s₁(t)s₂(t) will be explained. For example, the weight coefficient calculating circuit 115a is configured to derive a signal s₁*(t) by calculating the complex conjugate of the input signal s₁(t). In this situation, a signal sₙ*(t) is the conjugate complex number of the complex number represented by the signal sₙ(t).

Further, the weight coefficient calculating circuit 115a is configured to multiply the signal s₁*(t) by the input signal s₂(t) and to extract the phase ∠(s₁*(t)s₂(t)) of a signal s₁*(t)s₂(t) obtained as the result of the multiplication. In this situation, the phase ∠(s₁*(t)s₂(t)) is also a phase difference (a time difference) between the signal s₁(t) and the signal s₂(t). Further, the phase ∠(s₁*(t)s₂(t)) is also a correlation, a correlation coefficient, phase information, and an instantaneous phase value between the signal s₁(t) and the signal s₂(t).

Subsequently, the weight coefficient calculating circuit 115a is configured to calculate a weight coefficient in accordance with the phase ∠(s₁*(t)s₂(t)). For example, the weight coefficient calculating circuit 115a is configured to calculate the weight coefficient that decreases as the phase ∠(s₁*(t)s₂(t)) increases. In a specific example, the weight coefficient calculating circuit 115a is configured to calculate a weight coefficient w(θ(t)) by using Expression (1) presented below:

w(θ(t))=1−exp(−α|θ|) (1)

In Expression (1), 8(t) denotes the phase ∠(s₁*(t)s₂(t)). In other words, the following is true: the weight coefficient w(θ(t))=a weight coefficient w (∠(s₁*(t)s₂(t))). Further, in Expression (1), θ also denotes the phase ∠(s₁*(t)s₂(t)). Further, in Expression (1), α denotes a coefficient used for adjusting the magnitude of the weight coefficient w(θ(t)).

Further, the weight coefficient calculating circuit 115a is configured to output the calculated weight coefficient w (∠(s₁*(t)s₂(t))) to the multiplier 116a.

Similarly, the weight coefficient calculating circuit 115b is configured to calculate a weight coefficient w (∠(s₂*(t)s₃(t))) to be applied to the signal s₂(t)s₃(t), by using the signal s₂(t) and the signal s₃(t), while using the same method as the method used by the weight coefficient calculating circuit 115a to calculate the weight coefficient w(θ(t)) by using the signal s₁(t) and the signal s₂(t). Further, the weight coefficient calculating circuit 115b is configured to output the calculated weight coefficient w (∠(s₂*(t)s₃(t))) to the multiplier 116b.

Also, the weight coefficient calculating circuit 115c is configured to calculate a weight coefficient w (∠(s₃*(t)s₁(t))) to be applied to the signal s₃(t)s₁(t), by using the signal s₃(t) and the signal s₁(t), while using the same method as the method used by the weight coefficient calculating circuit 115a to calculate the weight coefficient w(θ(t)) by using the signal s₁(t) and the signal s₂(t). Further, the weight coefficient calculating circuit 115c is configured to output the calculated weight coefficient w (∠(s₃*(t)s₁(t))) to the multiplier 116c.

As explained above, the weight coefficient calculating circuits 115 are configured to calculate the weight coefficients on the basis of the correlations between the reception signals multiplied by each other. Further, the weight coefficient calculating circuits 115 are configured to calculate the weight coefficients on the basis of the phase information between the delayed signals multiplied by each other. The weight coefficient calculating circuits 115 are each an example of a weight calculating unit.

After that, the multiplier 116a is configured to multiply the signal s₁(t)s₂(t) by the weight coefficient w (∠(s₁*(t)s₂(t))) and to output a signal s₁′(t) obtained as the result of the multiplication to the adder 117. In this situation, as explained above, the weight coefficient w (∠(s₁*(t)s₂(t))) is a value that decreases as the phase difference (the time difference) between the signal s₁(t) and the signal s₂(t) increases. Accordingly, the multiplier 116a is able to output, to the adder 117, the signal s′(t) of which a rate of contribution to the ultrasound image data decreases as the phase difference (the time difference) between the signal s₁(t) and the signal s₂(t) increases. Consequently, the ultrasound diagnosis apparatus 1 according to the present embodiment is able to inhibit the occurrence of the speckle patterns, when the beamforming implementing the DMAS method which involves the multiplications and the addition of the signals is used as the beamforming method.

Similarly, the multiplier 116b is configured to multiply the signal s₂(t)s₃(t) by the weight coefficient w (∠(s₂*(t)s₃(t))) and to output a signal s₂′(t) obtained as the result of the multiplication to the adder 117. Also, the multiplier 116c is configured to multiply the signal s₃(t)s₁(t) by the weight coefficient w (∠(s₃*(t)s₁(t))) and to output a signal s₃′(t) obtained as the result of the multiplication to the adder 117.

As explained above, the multipliers 116 are configured to apply the weight coefficients to the signals obtained as the results of the multiplications. The multipliers 116 are each an example of an applying unit.

The adder 117 is configured to calculate the sum of all the input signals as a signal y(t). In other words, the adder 117 is configured to calculate the sum of the signal s₁′(t), the signal s₂′(t), and the signal s₃′(t) as the signal y(t). After that, the adder 117 is configured to store the signal y(t) into the buffer memory 120 as reflected-wave data.

As explained above, the multipliers 114 are configured to multiply the delayed signals from the mutually-different elements, so that the adder 117 adds together the signals obtained as the results of the multiplications. The multipliers 114 and the adder 117 are examples of a multiplying and adding unit.

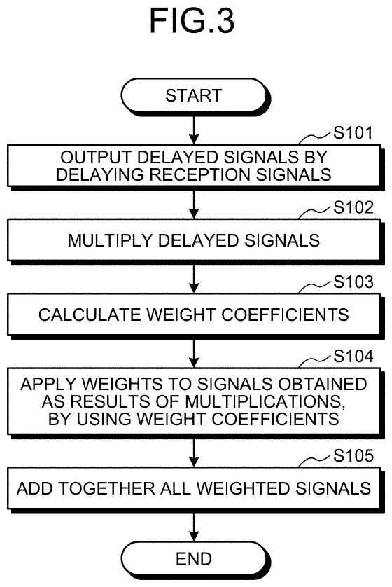

FIG. 3 is a flowchart illustrating an example of a flow in a process performed by the beam former implementing the DMAS method according to the first embodiment.

### Step S101:

As illustrated in FIG. 3, at step S101, the delay circuits 113 delay the IQ signals serving as reception signals and further output the delayed signals obtained by delaying the IQ signals.

### Step S102:

Subsequently, at step S102, with respect to two delayed signals output from mutually-different elements, each of the multipliers 114 multiplies one of the delayed signals by the other delayed signal and outputs the signal obtained as the result of the multiplication to a corresponding one of the multipliers 116.

### Step S103:

Subsequently, at step S103, each of the weight coefficient calculating circuits 115 calculates the weight coefficient w(θ(t)) and further outputs the weight coefficient w(θ(t)) to a corresponding one of the multipliers 116.

### Step S104:

After that, at step S104, each of the multipliers 116 applies a weight to the signal output from a corresponding one of the multipliers 114, by multiplying the signal output from the corresponding one of the multipliers 114 by the weight coefficient w(θ(t)) output from a corresponding one of the weight coefficient calculating circuits 115 and further outputs the weighted signal to the adder 117.

### Step S105:

Subsequently, at step S105, the adder 117 calculates the sum of the signals output from all the multipliers 116 as the signal y(t) and stores the calculated signal y(t) into the buffer memory 120 as the reflected-wave data. The process illustrated in FIG. 3 is thus ended.

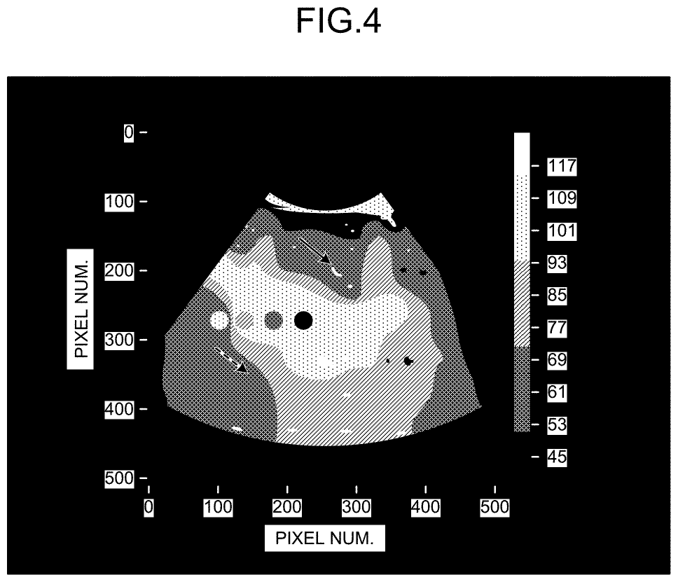

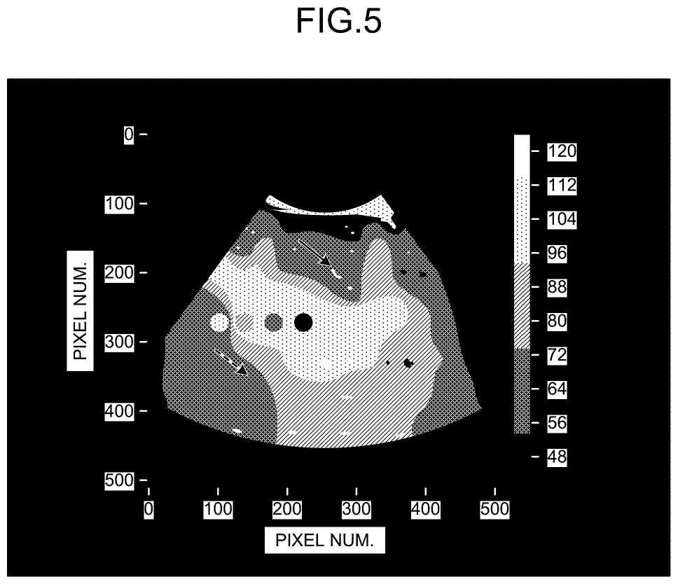

FIG. 4 is a drawing illustrating an example of an ultrasound image based on ultrasound image data obtained by implementing a conventional DMAS method. FIG. 5 is a drawing illustrating an example of an ultrasound image based on ultrasound image data generated by the ultrasound diagnosis apparatus 1 according to the first embodiment. In the ultrasound image presented in FIG. 4, relatively strong speckle patterns are present as indicated by the two arrows. In contrast, in the ultrasound image presented in FIG. 5, differences in the darkness (strength) of the speckle patterns are smaller than those in the ultrasound image presented in FIG. 4, as indicated by the two arrows. Consequently, according to the present embodiment, it is possible to inhibit the occurrence of the speckle patterns.

The ultrasound diagnosis apparatus 1 according to the first embodiment has thus been explained. By using the ultrasound diagnosis apparatus 1 according to the first embodiment, it is possible, as explained above, to inhibit the occurrence of the speckle patterns, when the beamforming implementing the DMAS method which involves the multiplications and the addition of the signals is used as the beamforming method.

### A Modification Example of First Embodiment

In the first embodiment described above, it is also possible to perform various types of processes on the signals output from the multipliers 114. Thus, this modification example will be explained as a modification example of the first embodiment. In the description of the modification example of the first embodiment, differences from the first embodiment will primarily be explained. Explanations of some of the configurations that are the same as those in the first embodiment may be omitted.

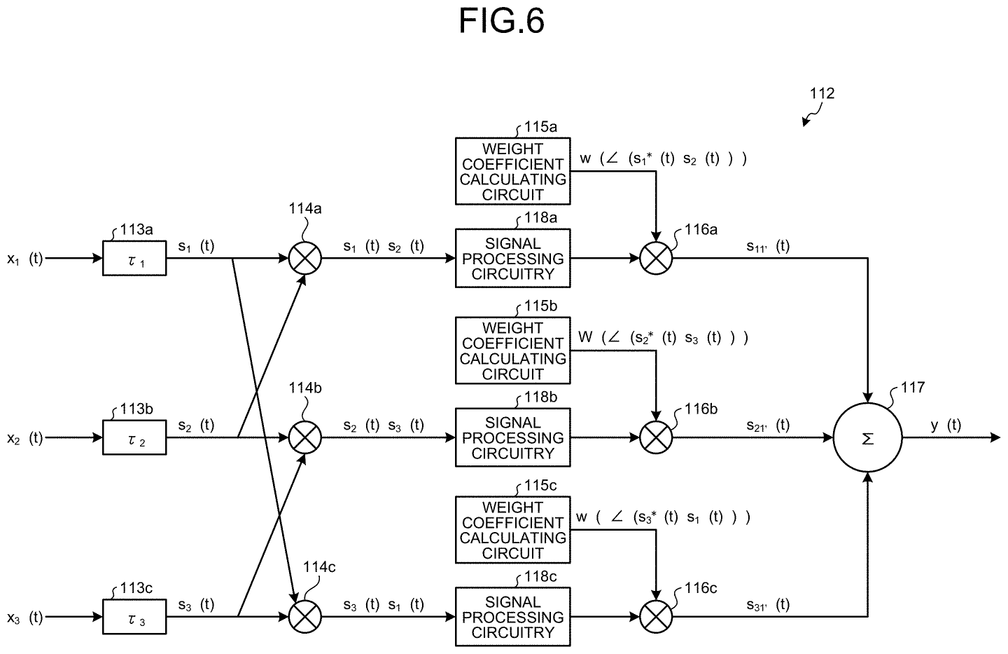

FIG. 6 is a diagram illustrating an exemplary configuration of a beam former included in the reception circuit 112 according to the modification example of the first embodiment and configured to implement beamforming using the DMAS method. The beam former according to the modification example of the first embodiment is different from the beam former according to the first embodiment illustrated in FIG. 2 for including three pieces of signal processing circuitry 118a to 118c.

When not being distinguished from one another, the three pieces of signal processing circuitry 118a to 118c will be referred to as “signal processing circuitry 118”. For example, one piece of signal processing circuitry 118 is provided with respect to each of the channels. To the signal processing circuitry 118, signals sᵢ(t)sⱼ(t) (where i, j=1, 2, 3; i≠j) output from the multipliers 114 are input. Further, by using the input signals sᵢ(t)sⱼ(t), the signal processing circuitry 118 is configured to calculate a signal sᵢ₁(t) by using Expression (2) presented below:

sᵢ₁(t)=sign(sᵢ(t)sⱼ(t))·|sᵢ(t)sⱼ(t)|^{1/2} (2)

In Expression (2), sign(sᵢ(t)sⱼ(t)) denotes a mathematical function that outputs the polarity (positive or negative) of the signal sᵢ(t)sⱼ(t), as described in, for example, Non-Patent Literature (The Delay Multiply and Sum Beamforming Algorithm in Ultrasound B-mode Medical Imaging, IEEE Transaction 2015).

For example, the signal processing circuitry 118a is configured to calculate a signal s₁₁(t) by using the signal s₁(t)s₂(t) output from the multiplier 114a. After that, the signal processing circuitry 118a is configured to output the signal s₁₁(t) to the multiplier 116a. Similarly, the signal processing circuitry 118b is configured to calculate a signal s₂₁(t) by using the signal s₂(t)s₃(t) output from the multiplier 114b. The signal processing circuitry 118c is configured to calculate a signal s₃₁(t) by using the signal s₃(t)s₁(t) output from the multiplier 114c. After that, the signal processing circuitry 118b is configured to output the signal s₂₁(t) to the multiplier 116b. The signal processing circuitry 118c is configured to output the signal s₃₁(t) to the multiplier 116c.

Further, the multiplier 116a is configured to multiply the signal sa (t) by the weight coefficient w (∠(s₁*(t)s₂(t))) and to output a signal s₁₁′(t) obtained as the result of the multiplication to the adder 117.

Similarly, the multiplier 116b is configured to multiply the signal s₂₁(t) by the weight coefficient w (∠(s₂*(t)s₃(t))) and to output a signal s₂₁′(t) obtained as the result of the multiplication to the adder 117. Also, the multiplier 116c is configured to multiply the signal s₃₁(t) by the weight coefficient w (∠(s₃*(t)s₁(t))) and to output a signal s₃₁′(t) obtained as the result of the multiplication to the adder 117.

The adder 117 is configured to calculate the sum of the signal s₁₁′(t), the signal s₂₁′(t), and the signal s₃₁′(t) as a signal y(t). After that, the adder 117 is configured to store the signal y(t) into the buffer memory 120 as reflected-wave data.

The ultrasound diagnosis apparatus 1 according to the modification example of the first embodiment has thus been explained. By using the ultrasound diagnosis apparatus 1 according to the modification example of the first embodiment, it is possible to achieve the same advantageous effects as those of the ultrasound diagnosis apparatus 1 according to the first embodiment.

### Second Embodiment

In the first embodiment, the example was explained in which the IQ signals obtained by the quadrature detection circuit are input to the multipliers 114; however, the ultrasound diagnosis apparatus 1 may be configured so that sub-aperture signals are input to the multipliers 114. Thus, this embodiment will be explained as a second embodiment. In the description of the second embodiment, differences from the first embodiment will primarily be explained. Explanations of some of the configurations that are the same as those in the first embodiment may be omitted.

In the second embodiment, the ultrasound probe 101 includes as many elements as 3k (where k is a natural number of 2 or larger), for example. Further, the 3k elements are divided in correspondence with three sub-apertures. In other words, each of the sub-apertures is structured with k elements. Accordingly, the 3k elements include a plurality of (three) element groups. Each of the element groups is structured with k elements. Further, to identify the three sub-apertures, a sub-aperture number m (where m=1, 2, 3) is assigned to each of the sub-apertures. For example, sub-aperture number 1 is assigned to a first sub-aperture; sub-aperture number 2 is assigned to a second sub-aperture; and sub-aperture number 3 is assigned to a third sub-aperture. Further, in the second embodiment, a sub-aperture signal aₘ(t) is a signal obtained by adding together k reception signals (IQ signals) at the time t output from the k elements structuring the sub-aperture identified with the aperture number m.

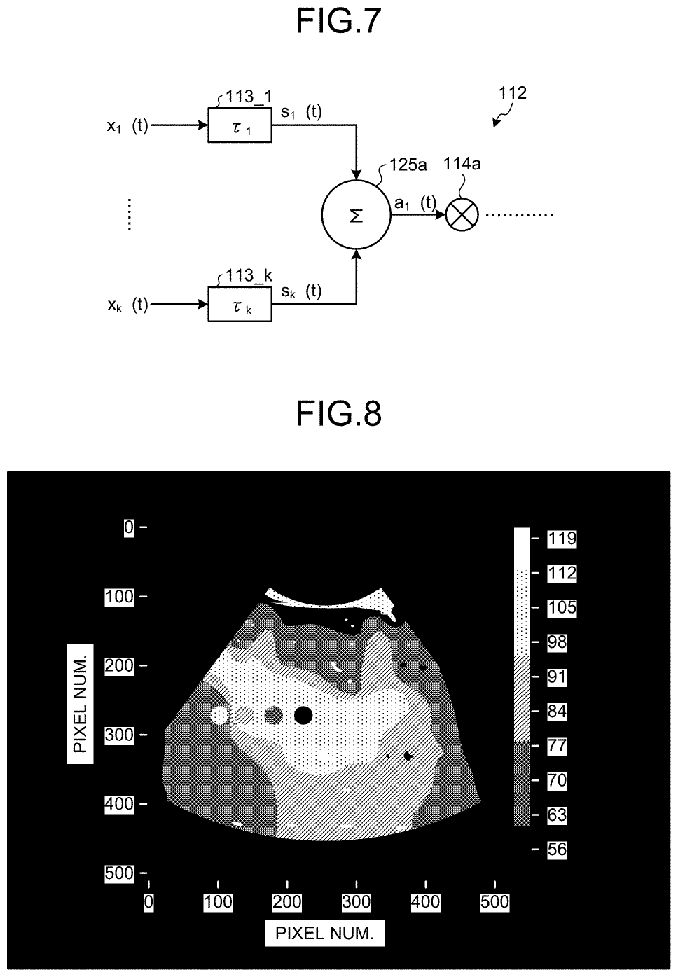

Next, an example in which a sub-aperture signal a₁(t) is input to the multiplier 114a will be explained, with reference to FIG. 7. By using the same configuration and method as described below, a sub-aperture signal a₂(t) and a sub-aperture signal a₃(t) are also generated.

FIG. 7 is a diagram illustrating an example of a partial configuration of a beam former according to the second embodiment. FIG. 7 illustrates the configuration preceding the multiplier 114a. As illustrated in FIG. 7, in the second embodiment, k delay circuits 113_1 to 113_k and one adder 125a are provided at a stage preceding the multiplier 114a.

In other words, the k delay circuits 113_1 to 113_k are provided in correspondence, respectively, with the k elements structuring the sub-aperture identified with sub-aperture number 1. As described herein, a sub-aperture beamforming unit corresponding to the sub-aperture identified with sub-aperture number 1 includes the k delay circuits 113_1 to 113_k and the one adder 125a. The sub-aperture beamforming unit is configured to output the sub-aperture signal a₁(t) by performing a delay-and-sum process on reception signals output from the element group structured with the k elements. As illustrated in FIG. 7, IQ signals x_{g}(t) are input to delay circuits 113_g (where g is a natural number from 1 to k, inclusive). The delay circuits 113_g are configured to output the IQ signals x_{g}(t) each delayed by a time period τg. In other words, the delay circuits 113_g are configured to output IQ signals s_{g}(t) obtained by delaying the IQ signals x_{g}(t) by the time period τg, to the adder 125a.

The adder 125a is configured to calculate the sum of all the input signals as the sub-aperture signal a₁(t). In other words, the adder 125a is configured to calculate the sum of the k IQ signals x_{g}(t) as the sub-aperture signal a₁(t). After that, the adder 125a is configured to output the sub-aperture signal a₁(t) to the multiplier 114a (see FIG. 7), the multiplier 114c (see FIG. 2), the weight coefficient calculating circuit 115a (see FIG. 2), and the weight coefficient calculating circuit 115c (see FIG. 2).

Further, in the second embodiment, by using the same configuration and method as described above, a sub-aperture signal a₂(t) is also generated and input to the multiplier 114a, the multiplier 114b, the weight coefficient calculating circuit 115a, and the weight coefficient calculating circuit 115b. In addition, by using the same configuration and method as described above, a sub-aperture signal a₃(t) is also generated and input to the multiplier 114b, the multiplier 114c, the weight coefficient calculating circuit 115b, and the weight coefficient calculating circuit 115c.

Further, in the second embodiment, the same processes as described in the first embodiment are performed by using the sub-aperture signal a₁(t) in place of the signal s₁(t), using the sub-aperture signal a₂(t) in place of the signal s₂(t), and using the sub-aperture signal a₃(t) in place of the signal s₂(t). Accordingly, in the second embodiment, the multipliers 114 are configured to multiply the sub-aperture signals by each other in all the combinations among the element groups described above.

FIG. 8 is a drawing illustrating an example of an ultrasound image based on ultrasound image data generated by the ultrasound diagnosis apparatus 1 according to the second embodiment. In the ultrasound image presented in FIG. 8, differences in the darkness (strength) of the speckle patterns are smaller than those in the ultrasound image in FIG. 4 presented earlier. Consequently, according to the present embodiment, it is possible to inhibit the occurrence of the speckle patterns.

The ultrasound diagnosis apparatus 1 according to the second embodiment has thus been explained. The ultrasound diagnosis apparatus 1 according to the second embodiment is able to achieve the same advantageous effects as those in the first embodiment. Further, in the second embodiment, because the reception signals from the plurality of elements are collectively processed, it is possible to shorten the computation time in the beamforming.

### Third Embodiment

In the second embodiment, the example was explained in which the IQ signals x₁(t) to xₖ(t) are input to the delay circuits 113_1 to 113_k, respectively. However, it is also acceptable to input, to each of the delay circuits 113_1 to 113_k, a signal of a harmonic component extracted by implementing a pulse subtraction method (a pulse inversion method). In other words, the ultrasound diagnosis apparatus 1 may be configured to perform harmonic imaging by implementing the pulse subtraction method. Thus, this embodiment will be explained as a third embodiment. In the description of the third embodiment, differences from the second embodiment will primarily be explained. Explanations of some of the configurations that are the same as those in the second embodiment may be omitted.

In the third embodiment, the controlling circuitry 170 is configured to cause the elements in the ultrasound probe 101 to perform an ultrasound scan employing a set made up of transmission of a first ultrasound wave and transmission of a second ultrasound wave obtained by inverting the phase of the first ultrasound wave. Accordingly, in the third embodiment, the elements in the ultrasound probe 101 are configured to transmit the first ultrasound wave and to also transmit the second ultrasound wave obtained by inverting the phase of the first ultrasound wave. Further, the elements are configured to output a first reception signal by receiving a reflected wave of the first ultrasound wave and to also output a second reception signal by receiving a reflected wave of the second ultrasound wave.

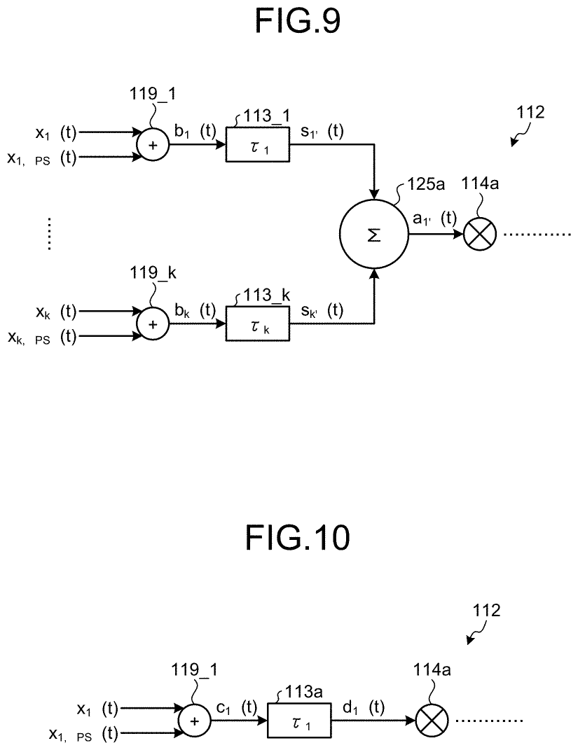

Next, with reference to FIG. 9, an example will be explained in which the IQ signals x_{g}(t) are IQ signals based on a first transmission ultrasound wave, whereas the IQ signals x_{g,PS}(t) are IQ signals based on a second transmission ultrasound wave. FIG. 9 is a diagram illustrating an example of a partial configuration of a beam former according to the third embodiment. In the third embodiment, a sub-aperture signal aₘ′(t) is a signal obtained by adding together k harmonic signals b_{g}(t) at the time t corresponding to the k elements structuring the sub-aperture identified with the sub-aperture number m. In the following sections, a configuration and a method for generating a sub-aperture signal a₁′(t) will be explained. By using the same configuration and method, a sub-aperture signal a₂′(t) and a sub-aperture signal a₃′(t) are also generated.

FIG. 9 illustrates a configuration preceding the delay circuits 113_g. As illustrated in FIG. 9, in the third embodiment, adders 119_g are provided at a stage preceding the delay circuits 113_g. In other words, k adders 119_1 to 119_k are provided in correspondence, respectively, with the k elements structuring the sub-aperture identified with sub-aperture number 1.

As illustrated in FIG. 9, the IQ signals x_{g}(t) and the IQ signals x_{g,PS}(t) are input to the adders 119_g. The adders 119_g are configured to generate harmonic signals b_{g}(t) by adding the IQ signals x_{g,PS}(t) to the IQ signals x_{g}(t). The IQ signals x_{q}(t) serve as an example of the first reception signal. Further, the IQ signals x_{g,PS}(t) serve as an example of the second reception signal. Further, the adders 119_g are configured to output the harmonic signals b_{g}(t) to the delay circuits 113_g. The adders 119_g serve as an example of a harmonic extracting unit.

The delay circuits 113_g are configured to output the harmonic signals b_{g}(t) each delayed by the time period τg. In other words, the delay circuits 113_g are configured to output harmonic signals sa′(t) obtained by delaying the harmonic signals b_{g}(t) by the time period τg, to the adder 125a.

The adder 125a is configured to calculate the sum of all the input signals as the sub-aperture signal a₁′(t). In other words, the adder 125a is configured to calculate the sum of the k harmonic signals s_{g}′(t) as the sub-aperture signal a₁′(t). Further, the adder 125a is configured to output the sub-aperture signal a₁′(t) to the multiplier 114a (see FIG. 9), the multiplier 114c (see FIG. 2), the weight coefficient calculating circuit 115a (see FIG. 2), and the weight coefficient calculating circuit 115c (see FIG. 2).

Further, in the third embodiment, by using the same configuration and method as described above, a sub-aperture signal a₂′(t) is also generated and input to the multiplier 114a, the multiplier 114b, the weight coefficient calculating circuit 115a, and the weight coefficient calculating circuit 115b. In addition, by using the same configuration and method as described above, a sub-aperture signal a₃′(t) is also generated and input to the multiplier 114b, the multiplier 114c, the weight coefficient calculating circuit 115b, and the weight coefficient calculating circuit 115c.

Further, in the third embodiment, the same processes as described in the second embodiment are performed by using the sub-aperture signal ad′(t) in place of the sub-aperture signal a₁(t), using the sub-aperture signal a₂′(t) in place of sub-aperture signal a₂(t), and using the sub-aperture signal aa′(t) in place of the sub-aperture signal a₃(t).

The ultrasound diagnosis apparatus 1 according to the third embodiment has thus been explained. The ultrasound diagnosis apparatus 1 according to the third embodiment is able to achieve the same advantageous effects as those in the first and the second embodiments. In the third embodiment, the example was explained in which the ultrasound diagnosis apparatus 1 is configured to extract the harmonic signals by implementing the pulse subtraction method while using the IQ signals x_{g}(t) and the IQ signals x_{g,PS}(t) and to subsequently delay the harmonic signals. However, in the third embodiment, the ultrasound diagnosis apparatus 1 may be configured to delay the IQ signals x_{g}(t) and the IQ signals x_{g,PS}(t) and to subsequently extract harmonic signals by implementing the pulse subtraction method while using the delayed IQ signals x_{g}(t) and IQ signals x_{g,PS}(t).

### Fourth Embodiment

In the first embodiment, the example was explained in which the IQ signals x₁(t) to x₃(t) are input to the delay circuits 113a to 113c, respectively. However, it is also acceptable to input a signal of a harmonic component extracted by implementing the pulse subtraction method to each of the delay circuits 113a to 113c. In other words, the ultrasound diagnosis apparatus 1 may be configured to perform harmonic imaging by implementing the pulse subtraction method. Thus, this embodiment will be explained as a fourth embodiment. In the description of the fourth embodiment, differences from the first to the third embodiments described above will primarily be explained. Explanations of some of the configurations that are the same as those in the first to the third embodiments may be omitted.

In the fourth embodiment, the ultrasound diagnosis apparatus 1 is configured to implement an ultrasound beamforming method by which harmonic signals from mutually-different elements are multiplied by each other, so that signals obtained as the results of the multiplications are added together. Similarly to the third embodiment, in the fourth embodiment, the controlling circuitry 170 is configured to cause the elements in the ultrasound probe 101 to perform an ultrasound scan employing a set made up of the transmission of the first ultrasound wave and the transmission of the second ultrasound wave obtained by inverting the phase of the first ultrasound wave. Accordingly, similarly to the third embodiment, in the fourth embodiment also, the elements in the ultrasound probe 101 are configured to transmit the first ultrasound wave and to also transmit the second ultrasound wave obtained by inverting the phase of the first ultrasound wave. Further, the elements are configured to output a first reception signal by receiving a reflected wave of the first ultrasound wave and to also output a second reception signal by receiving a reflected wave of the second ultrasound wave.

FIG. 10 is a diagram illustrating an example of a partial configuration of a beam former according to the fourth embodiment. In the following sections, a configuration and a method for generating a harmonic signal c₁(t) to be input to the delay circuit 113a will be explained. By using the same configuration and method, a harmonic signal c₂(t) and a harmonic signal c₃(t) are also generated. The harmonic signal c₂(t) is a signal to be input to the delay circuit 113b. The harmonic signal c₃(t) is a signal to be input to the delay circuit 113c.

FIG. 10 illustrates a configuration preceding the delay circuit 113a. As illustrated in FIG. 10, in the fourth embodiment, an adder 119_1 is provided at a stage preceding the delay circuit 113a. In other words, one adder 119 is provided in correspondence with each of the channels.

As illustrated in FIG. 10, the IQ signals x₁(t) and the IQ signals x_{1,PS}(t) are input to the adder 119_1. The adder 119_1 is configured to generate the harmonic signal c₁(t), by adding the IQ signals x_{1,PS}(t) to the IQ signals x₁(t). After that, the adder 119_1 is configured to output the harmonic signal c₁(t) to the delay circuit 113a.

The delay circuit 113a is configured to output the harmonic signal c₁(t) delayed by the time period 1l. In other words, the delay circuit 113a is configured to output a harmonic signal d₁(t) obtained by delaying the harmonic signal c₁(t) by the time period τ1, to the multiplier 114a (see FIG. 10), the multiplier 114c (see FIG. 2), the weight coefficient calculating circuit 115a (see FIG. 2), and the weight coefficient calculating circuit 115c (see FIG. 2).

Further, in the fourth embodiment, by using the same configuration and method as described above, a harmonic signal c₂(t) is also generated and input to the multiplier 114a, the multiplier 114b, the weight coefficient calculating circuit 115a, and the weight coefficient calculating circuit 115b. In addition, by using the same configuration and method as described above, a harmonic signal c₃(t) is also generated and input to the multiplier 114b, the multiplier 114c, the weight coefficient calculating circuit 115b, and the weight coefficient calculating circuit 115c. In this situation, to the multiplier 114a, the multiplier 114b, the weight coefficient calculating circuit 115a, and the weight coefficient calculating circuit 115b, a harmonic signal d₂(t) obtained as a result of the delay circuit 113b delaying the harmonic signal c₂(t) is input. Also, to the multiplier 114b, the multiplier 114c, the weight coefficient calculating circuit 115b, and the weight coefficient calculating circuit 115c, a harmonic signal d₂(t) obtained as a result of the delay circuit 113c delaying the harmonic signal c₃(t) is input.

After that, in the fourth embodiment, the same processes as described in the first embodiment are performed by using the harmonic signal d₁(t) in place of the IQ signals s₁(t), using the harmonic signal d₂(t) in place of the IQ signals s₂(t), and using the harmonic signal d₃(t) in place of the IQ signals s₃(t).

The ultrasound diagnosis apparatus 1 according to the fourth embodiment has thus been explained. The ultrasound diagnosis apparatus 1 according to the fourth embodiment is able to achieve the same advantageous effects as those in the first to the third embodiments. In the fourth embodiment, similarly to the third embodiment, the example was explained in which the ultrasound diagnosis apparatus 1 is configured to extract the harmonic signals by implementing the pulse subtraction method while using the IQ signals x_{g}(t) and the IQ signals x_{g,PS}(t) and to subsequently delay the harmonic signals. However, in the fourth embodiment, similarly to the third embodiment, the ultrasound diagnosis apparatus 1 may be configured to delay the IQ signals x_{g}(t) and the IQ signals x_{g,PS}(t) and to subsequently extract harmonic signals by implementing the pulse subtraction method while using the delayed IQ signals x_{g}(t) and IQ signals x_{g,PS}(t).

Further, the programs executed by the one or more processors are provided as being incorporated, in advance, into a Read-Only Memory (ROM), a storage circuit, or the like. Alternatively, the programs may be provided as being recorded on a non-transitory computer-readable recording medium such as a Compact Disk Read-Only Memory (CD-ROM), a Flexible Disk (FD), a Compact Disk Recordable (CD-R), a Digital Versatile Disk (DVD), or the like, in a file in a format that is installable or executable for these devices. Further, the programs may be stored in a computer connected to a network such as the Internet, so as to be provided or distributed as being downloaded via the network. For example, the programs are structured with modules including the processing functions described above. In the actual hardware, as a result of a CPU reading and executing the programs from a recording medium such as a ROM, the modules are loaded into a main storage device so as to be generated in the main storage device.

According to at least one aspect of the embodiments described above, it is possible to inhibit the occurrence of the speckle patterns, when the beamforming implementing the DMAS method which involves the multiplications and the addition of the signals is used as the beamforming method.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus that implements an ultrasound beamforming method by which, among a plurality of reception signals output from a plurality of elements, reception signals from mutually-different elements are multiplied by each other, so that signals obtained as results of the multiplications are added together, the ultrasound diagnosis apparatus comprising processing circuitry configured:
to calculate a weight coefficient on a basis of a correlation between the multiplied reception signals; and
to apply the weight coefficient to the signals obtained as the results of the multiplications.

2. The ultrasound diagnosis apparatus according to claim 1, wherein
the processing circuitry is configured:
to output a plurality of delayed signals delayed by applying a delay time period corresponding to a position of a focal point to the plurality of reception signals output from the plurality of elements;
to multiply the delayed signals from the mutually-different elements by each other, so that the signals obtained as the results of the multiplications are added together; and
to calculate the weight coefficient on a basis of phase information between the multiplied delayed signals.

3. The ultrasound diagnosis apparatus according to claim 2, wherein
the plurality of elements include a plurality of element groups, and
the processing circuitry is configured:
to output a sub-aperture signal with respect to each of the element groups by performing a delay-and-sum process on the reception signals output from each of the element groups;
to multiply the sub-aperture signals by each other in all combinations among the element groups so that the signals obtained as the results of the multiplications are added together; and
to calculate the weight coefficient on the basis of phase information between the multiplied sub-aperture signals.

4. The ultrasound diagnosis apparatus according to claim 3, wherein the processing circuitry is configured to calculate the weight coefficient on a basis of an instantaneous phase value between the multiplied delayed signals serving as the phase information between the multiplied delayed signals.

5. The ultrasound diagnosis apparatus according to claim 3, wherein the processing circuitry is configured to calculate the weight coefficient on a basis of a correlation coefficient between the multiplied delayed signals serving as the phase information between the multiplied delayed signals.

6. The ultrasound diagnosis apparatus according to claim 1, wherein
each of the plurality of elements is configured to transmit a first ultrasound wave and a second ultrasound wave obtained by inverting a phase of the first ultrasound wave and configured to output a first reception signal by receiving a reflected wave of the first ultrasound wave and to output a second reception signal by receiving a reflected wave of the second ultrasound wave, and
the processing circuitry is configured:
to extract a harmonic signal by adding the second reception signal to the first reception signal output from each of the plurality of elements; and
to implement the ultrasound beamforming method by which the harmonic signals from the mutually-different elements are multiplied by each other so that the signals obtained as the results of the multiplications are added together.

7. The ultrasound diagnosis apparatus according to claim 2, wherein
the plurality of elements include a plurality of element groups,
each of the plurality of elements is configured to transmit a first ultrasound wave and a second ultrasound wave obtained by inverting a phase of the first ultrasound wave and configured to output a first reception signal by receiving a reflected wave of the first ultrasound wave and to output a second reception signal by receiving a reflected wave of the second ultrasound wave, and
the processing circuitry is configured:
to extract a harmonic signal by adding the second reception signal to the first reception signal output from each of the plurality of elements;
to output a sub-aperture signal with respect to each of the element groups by performing a delay-and-sum process on the harmonic signals;
to multiply the sub-aperture signals by each other in all combinations among the element groups so that the signals obtained as the results of the multiplications are added together; and
to calculate the weight coefficient on the basis of phase information between the multiplied sub-aperture signals.

8. A non-transitory computer-readable recording medium having recorded thereon a program, wherein
a computer is configured to implement an ultrasound beamforming method by which, among a plurality of reception signals output from a plurality of elements, reception signals from mutually-different elements are multiplied by each other, so that signals obtained as results of the multiplications are added together, and
the program causes the computer to perform:
calculating a weight coefficient on a basis of a correlation between the multiplied reception signals; and
applying the weight coefficient to the signals obtained as the results of the multiplications.
